(19) United States
(12) Patent Application Publication
(10) Pub. No.: US 2008/0193894 A1
(43) Pub. Date: Aug. 14, 2008

(54) Mouth camera device

(76) Inventor: Neng-Wei Wu, Hsinchu City (TW)

Correspondence Address: Hershkovitz & Associates, LLC, 2845 Duke Street, Alexandria, VA, 22314, US

(21) Appl. No.: 11/705,465

(22) Filed: Feb. 13, 2007

(51) Int. Cl. A61B 1/24

(52) U.S. Cl. 433/30

(57) ABSTRACT

A mouth camera device has a camera and a mouth mirror. The camera has a handle, a lens, a photo sensor assembly, a PCB assembly and a computer output port. The computer output port is connected to a computer. The mouth mirror is mounted detachably in the handle. The camera may catch and transmit static or dynamic images to the computer so a diagnostician and a patient may clearly and easily watch a condition inside the patient's mouth cavity. Diagnosing the mouth cavity is convenient and timesaving.

### BACKGROUND OF THE INVENTION

1. Field of the Invention

The present invention relates to an oval diagnostic device, and more particularly to a mouth camera device that has a mouth mirror and a camera.

2. Description of Related Art

Mouth mirrors are widely used in oral diagnosis to reflect an interior of a patient's oral cavity so a diagnostician such as a dentist or an ear-nose-and-throat doctor may observe and diagnose mouth tissue through the mouth mirrors.

A conventional mouth mirror has a handle and a mirror head. The handle is longitudinal and has a front end. The mirror head is mounted on the front end of the handle and has a mirror surface. When the mirror is used, the mirror head is inserted into a patient's mouth cavity to reflect oral tissue. However, a dimension of the mirror head is as small as a coin in order to be put easily into the mouth cavity and may reflect a limited area of the mouth tissue so watching the mirror head is very strenuous to a diagnostician. Furthermore, the patient cannot watch his/her own mouth tissue through mouth mirror so the patient may not be aware of how the condition inside the mouth cavity is.

To overcome the shortcomings, the present invention provides a mouth camera device to mitigate or obviate the aforementioned problems.

### SUMMARY OF THE INVENTION

The main objective of the invention is to provide a mouth camera device that has a mouth mirror and a camera that receives reflected images from the mouth mirror and transmits the reflected images to an external display.

A mouth camera device in accordance with the present invention comprises a camera and a mouth mirror. The camera has a handle, a lens, a photo sensor assembly, a PCB assembly and a computer output port. The computer output port may connect to a computer. The mouth mirror is mounted detachably in the handle. The camera may catch and transmit static or dynamic images to the computer so a diagnostician and a patient may clearly and easily watch a condition inside the patient's mouth cavity.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a mouth camera device in accordance with the present invention;

FIG. 2 is an exploded perspective view of the mouth camera device in FIG. 1;

FIG. 3 is a side view of the mouth camera device in FIG. 1 with one of the casing halves removed; and

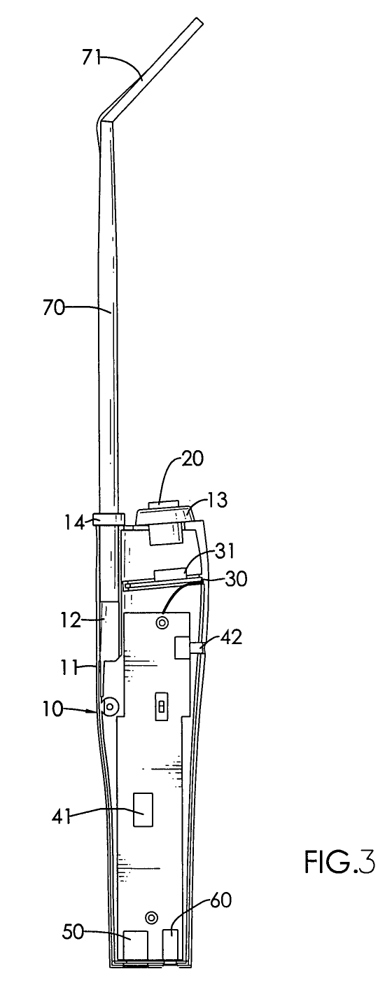

FIG. 4 is a perspective view of the mouth camera device in FIG. 1 with a sleeve.

### DETAILED DESCRIPTION OF THE PREFERRED EMBODIMENT

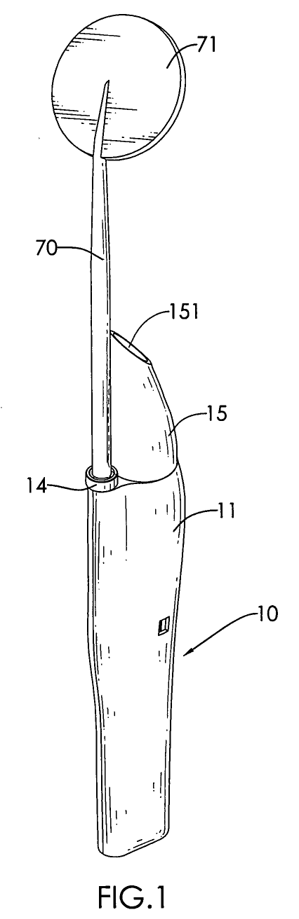

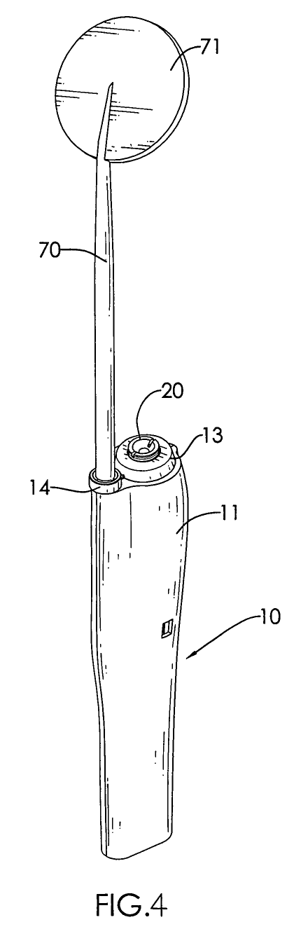

With reference to FIGS. 1 and 4, a mouth camera device in accordance with the present invention comprises a camera and a mouth mirror.

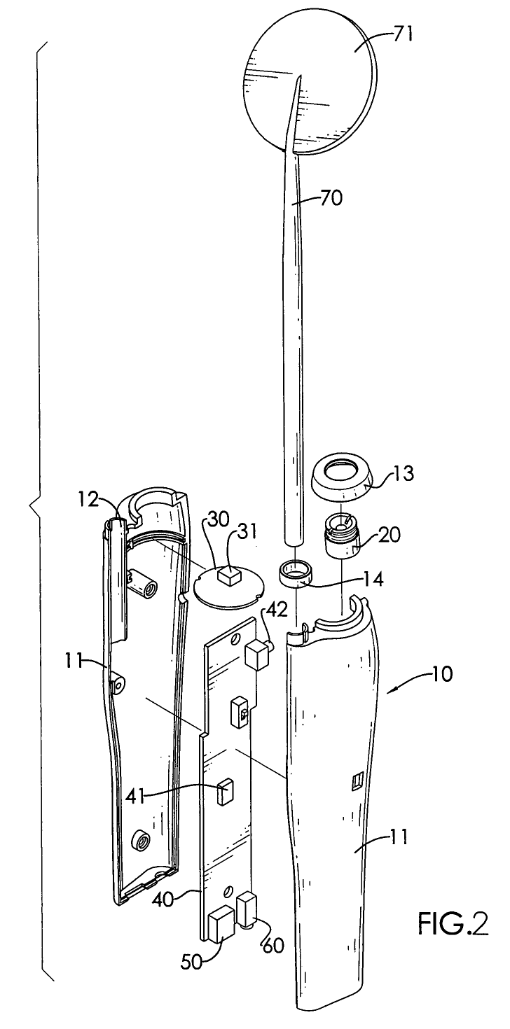

With further reference to FIG. 2, the camera has a handle (10), a lens (20), a photo sensor assembly, a printed circuit board (PCB) assembly, a computer output port (50) and a video output terminal (60).

The handle (10) comprises two casing halves (11) and has an axis, a front end, a cavity, a mounting slot (12), two mounting collars (13, 14) and a sleeve. The cavity is defined in the handle (10) and has a front opening and a mounting flange. The front opening is defined in the front end. The mounting flange is annular and is formed on the front end around the front opening. The mounting slot (12) is defined in the handle (10) and has a front opening and a mounting flange. The front opening is defined in the front end. The mounting flange of the mounting slot (12) is annular and is formed on the front end. One mounting collar (13) is mounted around the mounting flange on the cavity and the other mounting collar (14) is mounted around the mounting flange on the mounting slot (12) to securely combine the casing halves (11) together. The sleeve (15) is mounted detachably around the mounting flange of front opening of the cavity and may extends in a patient's mouth cavity so the patient's lips or teeth would not block the lens (20) described hereafter. The sleeve (15) may be tapered forward from the front opening in the handle (10).

The lens (20) is mounted in the front opening of the cavity and has an axis. The axis of the lens (20) intersects the axis of the handle (10) at an acute angle of 5°.

The photo sensor assembly is mounted in the cavity in the handle (10) and has a sensor PCB (30) and a photo sensor (31). The sensor PCB (30) is mounted in the cavity in the handle (10). The photo sensor (31) may be a charge-coupled device (CCD) or a complementary metal oxide semiconductor (CMOS), is mounted on the sensor PCB (30) and is aligned with the lens (20) to catch static or dynamic images through the lens (20).

The PCB assembly is mounted in the cavity in the handle (10), connects electrically to the photo sensor assembly and has a control PCB (40), a control circuit (41) and a camera shutter button (42). The control PCB (40) is mounted in the cavity in the handle (10) and connects electrically to the sensor PCB (30). The control circuit (41) is mounted on the control PCB (40) and processes and transforms the images from the photo sensor (31) to electrical image files. The camera shutter button (42) is mounted on the control PCB (40), extends out from the handle (10) and may be pressed to activate the control circuit (41).

The computer output port (50) is mounted on the control PCB (40), extends out from the handle (10), may be and universal serial bus (USB) port, an external serial advanced technology attachment (eSATA) port and may connect to computers through a cable.

The video output terminal (60) is mounted on the control PCB (40), may be an S-video terminal or an audio/video terminal and may connect to external televisions or monitors through a cable.

The mouth mirror is mounted detachably in the handle (10) and has a stem (70) and a mirror head (71). The stem (70) is mounted detachably in the mounting slot (12) and has a proximal end and a distal end. The mirror head (71) is mounted on the distal end of the stem (70), is aligned with the lens (20) and has a mirror surface aligned with the lens capable of reflecting images from a patient's mouth cavity to the lens (20).

When observing and diagnosing a patient's the mouth cavity, a diagnostician extends the stem (70) with the mirror head (71) and the sleeve (15) in the mouth cavity however the handle (10) is kept outside the mouth cavity. Therefore, the photo sensor assembly and the PCB assembly keep away from the spit in the mouth cavity and would not break easily. Furthermore, the sleeve (15) extend through the patient's lips and teeth to ensure that no obstruction is lies on a light path between the mirror head (71) and the lens (20). The images caught by the camera is transmitted to a display such as a monitor, a television or a computer monitor so the diagnostician and the patient may clearly and easily watch the condition in the mouth cavity through the display.

Because the camera may catch and transmit static or dynamic images to external displays so a diagnostician and a patient may clearly and easily watch a condition inside the patient's mouth cavity. Therefore, diagnosing the mouth cavity is convenient and timesaving.

Even though numerous characteristics and advantages of the present invention have been set forth in the foregoing description, together with details of the structure and function of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A mouth camera device comprising:
a camera having
a handle having
a front end;
a cavity defined in the handle and having a front opening defined in the front end; and
a mounting slot defined in the front end;
a lens mounted in the front opening of the cavity in the handle;
a photo sensor assembly is mounted in the cavity in the handle and having
a sensor printed circuit board (PCB) mounted in the cavity in the handle; and
a photo sensor mounted on the sensor PCB and aligned with the lens;
a PCB assembly mounted in the cavity in the handle, connecting electrically to the photo sensor assembly and having
a control PCB mounted in the cavity in the handle, connecting electrically to the sensor PCB;
a control circuit mounted on the control PCB and adapted to processing and transforming images from the photo sensor to electrical image files; and
a camera shutter button mounted on the control PCB, extending out from the handle and selectively activating the control circuit; and
a computer output port mounted on the control PCB, extending out from the handle and adapted to connect to a computer through a cable; and
a mouth mirror mounted detachably in the handle and having
a stem mounted detachably in the mounting slot in the handle and having a distal end; and
a mirror head mounted on the distal end of the stem and aligned with the lens to reflect images from a patient's mouth cavity to the lens.

2. The mouth camera device as claimed in claim 1, wherein the handle further has a sleeve mounted detachably on the front opening of the cavity.

3. The mouth camera device as claimed in claim 2, wherein the sleeve is tapered forward from the front opening in the handle.

4. The mouth camera device as claimed in claim 1 further comprising a video output terminal mounted on the control PCB and adapted to connect to a monitor.

5. The mouth camera device as claimed in claim 1, wherein:
the handle comprises two casing halves;
the cavity further has a mounting flange being annular and formed on the front end of the handle;
the mounting slot further has a mounting flange being annular and formed on the front end of the handle; and
the handle further has two mounting collars mounted respectively around the mounting flanges to securely combine the casing halves together.
